(19) United States
(12) Patent Application Publication
(10) Pub. No.: US 2014/0256820 A1
(43) Pub. Date: Sep. 11, 2014

(54) THERAPEUTIC AGENT FOR BLOOD-BRAIN BARRIER DISRUPTION SYNDROME

(71) Applicants: Fuyuko Takata, Fukuoka-shi (JP); Shinya Dohgu, Fukuoka-shi (JP); Yasufumi Kataoka, Fukuoka-shi (JP); Junichi Matsumoto, Fukuoka-shi (JP); Syuji Kaneshima, Fukuoka-shi (JP)

(72) Inventors: Fuyuko Takata, Fukuoka-shi (JP); Shinya Dohgu, Fukuoka-shi (JP); Yasufumi Kataoka, Fukuoka-shi (JP); Junichi Matsumoto, Fukuoka-shi (JP); Syuji Kaneshima, Fukuoka-shi (JP)

(73) Assignee: FUKUOKA UNIVERSITY, Fukuoka-shi, Fukuoka (JP)

(21) Appl. No.: 13/993,587

(22) PCT Filed: Dec. 12, 2011
(86) PCT No.: PCT/JP2011/079266

§ 371 Date: Jul. 31, 2013

(30) Foreign Application Priority Data
Dec. 13, 2010 (JP) 2010-277027

(51) Int. Cl. A61K 31/155

(52) U.S. Cl. CPC A61K 31/155
USPC 514/635; 564/233

(57) ABSTRACT

A medicinal agent or a pharmaceutical composition, each of which comprises a biguanides agent or a pharmaceutically acceptable salt thereof as an active ingredient, and which can potentiate blood-brain barrier functions including tight junction capability and transcellular transport capability of a brain capillary endothelial cell and therefore can treat blood-brain barrier dysfunction syndrome.

### TECHNICAL FIELD

The present invention relates to a therapeutic agent for blood-brain barrier dysfunction syndrome. More specifically, the present invention relates to a therapeutic agent for blood-brain barrier dysfunction syndrome comprising a biguanides agent or a pharmaceutically acceptable salt thereof as an active ingredient for treating or preventing various diseases by enhancing blood-brain barrier functions.

### BACKGROUND ART

The blood-brain barrier (BBB) is composed principally of cerebrovascular endothelial cells and separates circulating blood from the brain parenchyma. This barrier restricts the transport of substances such as drugs into the brain. In addition, the blood-brain barrier that is composed of cerebrovascular endothelial cells together with brain pericytes and glia cells maintains higher brain functions by forming a “cerebral neurovascular unit” that integrates network functions together with brain neurons. These blood-brain barrier dysfunctions are involved in the formation of various pathological conditions of brain dysfunction (Non Patent Literature 1).

The pathological conditions caused by blood-brain barrier dysfunction such as a decline in blood-brain barrier functions include, for example, (i) the increased amount of drugs transferred into the brain, (ii) cerebral edema attributed to the brain infiltration of serum albumin, (iii) encephalopathy attributed to leukocyte infiltration into the brain, and (iv) the defective cerebral clearance of biologically active peptides such as amyloid beta protein (Aβ). These pathological conditions cause (1) the occurrence of adverse reactions (side effects) of drugs and the onset or worsening of diseases attributed to blood-brain barrier dysfunction (hereinafter, these diseases are also collectively referred to as blood-brain barrier dysfunction syndrome), such as (2) cerebral infarction or cerebral trauma, (3) neurodegenerative disease in the brain, and (4) Alzheimer's disease.

Imatinib, a therapeutic agent for chronic myeloid leukemia or the like, is known to suppress blood-brain barrier permeability, thereby suppressing cerebral infarction (Non Patent Literature 2). Also, pranlukast, a therapeutic agent for bronchial asthma or the like, is known to serve as a vascular hyperpermeability-suppressing agent that acts on the lumens of capillary vessels and vascular endothelial cells constituting the lumen structure to prevent plasma components, blood cells, etc., in the capillary vessels from being leaked into tissue (Patent Literature 1).

However, any pharmaceuticals agent for treating blood-brain barrier dysfunction syndrome by enhancing blood-brain barrier functions has not yet been in actual use.

### CITATION LIST

### Patent Literature

Patent Literature 1: Japanese Patent Laid-Open No. 2009-221217

### Non Patent Literature

Non Patent Literature 1: Neuron, 2008, 57, 178-201
Non Patent Literature 2: Nature Medicine, 2008, July, 14(7), 731-737
Non Patent Literature 3: Nicolas W., Florence M., Sylvie C., Pierre-Olivier C., “The blood-brain barrier in brain hoeostasis and neurological diseases”, Biochimica et Biophysica Acta, 2009, 1778, p. 842-857
Non Patent Literature 4: Nishioku T., Yamauchi A., Takata F., Watanabe T., Furusho K., Shuto H., Dohgu S., Kataoka Y., “Disruption of the blood-brain barrier in collagen-induced arthritic mice”, Neuroscience Letters. 2010, Oct. 4, 482(3), p. 208-211
Non Patent Literature 5: Yamauchi A., Dohgu S., Takata F., Watanabe T., Nishioku T., Matsumoto J., Ohkubo Y., Shuto H., Kataoka Y., “Partial hepatectomy aggravates cyclosporin A-induced neurotoxicity by lowering the function of the blood-brain barrier in mice”, Life Sciences, 2011, Mar. 14, 88(11-12), p. 529-534
Non Patent Literature 6: Dohgu S., Sumi N., Nishioku T., Takata F., Watanabe T., Naito M., Shuto H., Yamauchi A., Kataoka Y., “Cyclosporin A induces hyperpermeability of the blood-brain barrier by inhibiting autocrine adrenomedullin-mediated up-regulation of endothelial barrier function”, European Journal of Pharmacology, 2010, Oct. 10, 644(1-3), p. 5-9
Non Patent Literature 7: Dohgu S., Nishioku T., Sumi N., Takata F., Nakagawa S., Naito M., Tsuruo T., Yamauchi A., Shuto H., Kataoka Y., “Adverse effect of cyclosporin A on barrier functions of cerebral microvascular endothelial cells after hypoxia-reoxygenation damage in vitro”, Cellular and Molecular Neurobiology, 2007, November, 27(7), p. 889-899
Non Patent Literature 8: Takata F., Dohgu S., Yamauchi A., Sumi N., Nakagawa S., Naito M., Tsuruo T., Shuto H., Kataoka Y., “Inhibition of transforming growth factor-beta production in brain pericytes contributes to cyclosporin A-induced dysfunction of the blood-brain barrier”, Cellular and Molecular Neurobiology, 2007, May, 27(3), p. 317-328
Non Patent Literature 9: Yamauchi A., Shuto H., Dohgu S., Nakano Y., Egawa T., Kataoka Y., “Cyclosporin A aggravates electroshock-induced convulsions in mice with a transient middle cerebral artery occlusion”, Cellular and Molecular Neurobiology, 2005, August, 25(5), p. 923-928
Non Patent Literature 10: Dohgu S., Yamauchi A., Nakagawa S., Takata F., Kai M., Egawa T., Naito M., Tsuruo T., Sawada Y., Niwa M., Kataoka Y., “Nitric oxide mediates cyclosporine-induced impairment of the blood-brain barrier in cocultures of mouse brain endothelial cells and rat astrocytes”, European Journal of Pharmacology, 2004, Nov. 28, 505(1-3), p. 51-59
Non Patent Literature 11: Yamauchi A., Oishi R., Kataoka Y., “Tacrolimus-induced neurotoxicity and nephrotoxicity is ameliorated by administration in the dark phase in rats”, Cell Mol Neurobiol, 2004, October, 24(5), p. 695-704
Non Patent Literature 12: Fujisaki Y., Yamauchi A., Dohgu S., Sunada K., Yamaguchi C., Oishi R., Kataoka Y., “Cyclosporine A-increased nitric oxide production in the rat dorsal hippocampus mediates convulsions”, Life Sciences, 2002, Dec. 20, 72(4-5), p. 549-556
Non Patent Literature 13: Tominaga K., Kai M., Yamauchi A., Dohgu S., Toda K., Oishi R., Kataoka Y., “Subchronic treatment with cyclosporin A decreases the binding properties of the GABAA receptor in ovariectomized rats”, Life Sciences, 2002, Dec. 20, 72(4-5), p. 425-430
Non Patent Literature 14: Kanoski S E, Zhang Y., Zheng W., Davidson T L, “The effects of a high-energy diet on hippocampal function and blood-brain barrier integrity in the rat”, Journal of Alzheimer's Disease, 2010, January, 21(1), p. 207-219
Non Patent Literature 15: Ueno M, Sakamoto H, Tomimoto H, Akiguchi I, Onodera M, Huang C L, Kanenishi K., “Blood-brain barrier is impaired in the hippocampus of young adult spontaneously hypertensive rats.”, Acta Neuropathologica, 2004, June, 107(6), p. 532-538
Non Patent Literature 16: Honda M., Nakagawa S., Hayashi K., Kitagawa N., Tsutsumi K., Nagata I., Niwa M., “Adrenomedullin improves the blood-brain barrier function through the expression of claudin-5”, Cellular and Molecular Neurobiology, 2006, 26(2), p. 109-118
Non Patent Literature 17: Jin R., Yang G., Li G., “Molecular insights and therapeutic targets for blood-brain barrier disruption in ischemic stroke: critical role of matrix metalloproteinases and tissue-type plasminogen activator”, Neurobiol of Disease, 2010, June, 38(3), p. 376-385
Non Patent Literature 18: Abu Fanne R., Nassar T., Yarovoi S., Rayan A., Lamensdorf I., Karakoveski M., Vadim P., Jammal M., Cines D. B., Higazi A. A., “Blood-brain barrier permeability and tPA-mediated neurotoxicity”, Neuropharmacology, 2010, June, 58(7), p. 972-980

### SUMMARY OF INVENTION

### Technical Problem

An object of the present invention is to provide a pharmaceutical composition for treating or preventing various diseases by enhancing blood-brain barrier functions.

### Solution to Problem

As a result of conducting diligent studies, the present inventor has completed the present invention by finding that a medicament comprising a biguanides agent or a pharmaceutically acceptable salt thereof as an active ingredient or a pharmaceutical composition containing the medicament enhances blood-brain barrier functions including tight junction between cerebrovascular endothelial cells, transcellular transport, etc., thereby suppressing the leakage of albumin present in brain capillary or a tight junction marker fluorescein sodium into brain tissue.

In this context, the term “enhancement” used in the present specification and claims means enhancement of a function decreased congenitally or after birth as well as enhancement of a normal function and further means enhancement of a function by “reconstruction” that improves the function decreased congenitally or after birth. This term also means “suppression” of a decline in function.

In addition, the term also means “promotion” of enhancement of a function through an external or internal factor.

Thus, the enhancement of blood-brain barrier functions including tight junction, transcellular transport, etc. shall be interpreted not only as being the enhancement of the blood-brain barrier functions but also as including the reconstruction of the blood-brain barrier functions, the suppression of a decline in the blood-brain barrier functions, and the promotion of the enhancement of the blood-brain barrier functions, the reconstruction thereof, and the suppression of the decline.

Specifically, the present application provides a therapeutic agent for blood-brain barrier dysfunction syndrome which is a medicament or a pharmaceutical composition comprising a biguanides agent or a pharmaceutically acceptable salt thereof as an active ingredient for enhancing a blood-brain barrier function.

More specifically, the present application provides a therapeutic agent for blood-brain barrier dysfunction syndrome according to the present invention for preventing worsening or recurrence of cerebral infarction. The present application also provides a therapeutic agent for blood-brain barrier dysfunction syndrome according to the present invention for preventing, suppressing, or ameliorating cerebral edema associated with cerebral infarction. The present application further provides a therapeutic agent for blood-brain barrier dysfunction syndrome according to the present invention for preventing, suppressing, or ameliorating encephalopathy associated with sepsis. The present application further provides a therapeutic agent for blood-brain barrier dysfunction syndrome according to the present invention for preventing, suppressing, or ameliorating leukocyte infiltration into the brain in multiple sclerosis. The present application further provides a therapeutic agent for blood-brain barrier dysfunction syndrome according to the present invention for preventing, suppressing, or ameliorating accumulation of amyloid beta protein (Aβ) in the brain in Alzheimer's disease.

In addition, the present application provides a therapeutic agent for blood-brain barrier dysfunction syndrome according to the present invention for preventing or suppressing drug transport into the brain, thereby preventing, suppressing, or ameliorating the adverse reaction that affects an animal or human central nervous system.

The present invention relates to the following items, though some items are already described above:

### (Item 1)

A medicament for enhancing a blood-brain barrier function, comprising a biguanides agent or a pharmaceutically acceptable salt thereof as an active ingredient.

### (Item 2)

The medicament according to any one of item 1, wherein said enhancement of the blood-brain barrier function is prevention, suppression, or promotion of the suppression of a decline in the blood-brain barrier function.

### (Item 3)

The medicament according to item 2, wherein said decline in the blood-brain barrier function is attributed to a disease selected from the group consisting of diabetes mellitus, obesity, hypertension, cerebral infarction, cerebral trauma, sepsis, multiple sclerosis, Alzheimer's disease, and combination thereof, or attributed to a substance inducing the central adverse reactions.

### (Item 4)

The medicament according to item 3, wherein said substance inducing the central adverse reactions is an immunosuppressive drug or a thrombolytic drug.

### (Item 5)

The medicament according to item 1, wherein the medicament is used in combination with a drug for preventing, suppressing, or ameliorating cerebral edema associated with cerebral infarction or cerebral trauma.

### (Item 6)

The medicament according to item 5, wherein said drug is a thrombolytic drug.

### (Item 7)

The medicament according to item 6, wherein said thrombolytic drug is a tissue plasminogen activator (t-PA).

### (Item 8)

The medicament according to item 1, wherein the medicament prevents or suppresses drug transport into the brain.

### (Item 9)

The medicament according to item 8, wherein said drug is a drug that possesses the unwanted effects on an animal or human central nervous system.

### (Item 10)

The medicament according to item 8 or 9, wherein the medicament prevents or suppresses said drug transport into the brain, thereby preventing, suppressing, or ameliorating an adverse reaction that affects the animal or human central nervous system.

### (Item 11)

The medicament according to item 10, wherein said adverse symptom is selected from tremor, convulsion, leukoencephalopathy, headache, sleepiness, disturbance of consciousness, abnormal behavior, delirium, hallucination, delusion, epilepsy, and combination thereof.

### (Item 12)

The medicament according to any one of items 1 to 11, wherein said biguanides agent is metformin or buformin.

### (Item 13)

The medicament according to item 12, wherein said biguanides agent or the pharmaceutically acceptable salt thereof is metformin hydrochloride or buformin hydrochloride.

### (Item 14)

The medicament according to any one of items 1 to 13, wherein a dose of the active ingredient is 125 to 3000 mg/day/person.

### (Item 15)

A method for preventing, suppressing, or ameliorating a disease wherein the disease is selected from the group consisting of tremor, convulsion, leukoencephalopathy, headache, sleepiness, disturbance of consciousness, abnormal behavior, delirium, hallucination, delusion, epilepsy, cerebral infarction, cerebral trauma, sepsis, Alzheimer's disease, and combination thereof, the method comprising the step of

administering a medicament according to item 1 to an individual in need thereof.

### (Item 16)

A method for treating blood-brain barrier dysfunction syndrome, comprising the step of

administering a medicament comprising a biguanides agent or a pharmaceutically acceptable salt thereof as an active ingredient to an individual in need thereof to enhance a blood-brain barrier function.

The present invention further relates to the following items, though some items are already described above:

### (Item 1a)

A therapeutic agent for blood-brain barrier dysfunction syndrome comprising a biguanides agent or a pharmaceutically acceptable salt thereof as an active ingredient for preventing or suppressing drug transport into the brain.

### (Item 2a)

The therapeutic agent for blood-brain barrier dysfunction syndrome according to item 1a, wherein said drug is a drug that possesses the unwanted effects on an animal or human central nervous system.

### (Item 3a)

The therapeutic agent for blood-brain barrier dysfunction syndrome according to item 1a or 2a, wherein the therapeutic agent prevents or suppresses said drug transport into the brain, thereby preventing, suppressing, or ameliorating an adverse reaction that affects the animal or human central nervous system.

### (Item 4a)

The therapeutic agent for blood-brain barrier dysfunction syndrome according to item 3a, wherein said the adverse reaction is selected from tremor, convulsion, leukoencephalopathy, headache, sleepiness, disturbance of consciousness, abnormal behavior, delirium, hallucination, delusion, epilepsy, and combination thereof.

### (Item 5a)

A therapeutic agent for blood-brain barrier dysfunction syndrome comprising a biguanides agent or a pharmaceutically acceptable salt thereof as an active ingredient for preventing, suppressing, or ameliorating cerebral edema associated with cerebral infarction or cerebral trauma.

### (Item 6a)

A therapeutic agent for blood-brain barrier dysfunction syndrome comprising a biguanides agent or a pharmaceutically acceptable salt thereof as an active ingredient for preventing, suppressing, or ameliorating encephalopathy associated with sepsis.

### (Item 7a)

A therapeutic agent for blood-brain barrier dysfunction syndrome comprising a biguanides agent or a pharmaceutically acceptable salt thereof as an active ingredient for preventing, suppressing, or ameliorating leukocyte infiltration into the brain in multiple sclerosis.

### (Item 8a)

A therapeutic agent for blood-brain barrier dysfunction syndrome comprising a biguanides agent or a pharmaceutically acceptable salt thereof as an active ingredient for preventing, suppressing, or ameliorating accumulation of amyloid beta protein (Aβ) in the brain in Alzheimer's disease.

### (Item 9a)

A therapeutic agent for blood-brain barrier dysfunction syndrome comprising a biguanides agent or a pharmaceutically acceptable salt thereof as an active ingredient for preventing or suppressing worsening or recurrence of cerebral infarction.

### (Item 10a)

A therapeutic agent for blood-brain barrier dysfunction syndrome comprising a biguanides agent or a pharmaceutically acceptable salt thereof as an active ingredient for enhancing a blood-brain barrier function.

### (Item 11a)

The therapeutic agent for blood-brain barrier dysfunction syndrome according to item 10a, wherein said enhancement of the blood-brain barrier function is prevention, suppression, or promotion of the suppression of a decline in the blood-brain barrier function.

### (Item 12a)

The therapeutic agent for blood-brain barrier dysfunction syndrome according to item 11a, wherein said decline in the blood-brain barrier function is attributed to a disease selected from the group consisting of diabetes mellitus, obesity, hypertension, and combination thereof.

### (Item 13a)

The therapeutic agent for blood-brain barrier dysfunction syndrome according to any one of items 1a to 12a, wherein said biguanides agent is metformin or buformin.

### (Item 14a)

The therapeutic agent for blood-brain barrier dysfunction syndrome according to item 13a, wherein said biguanides agent or the pharmaceutically acceptable salt thereof is metformin hydrochloride or buformin hydrochloride.

### (Item 15a)

The therapeutic agent for blood-brain barrier dysfunction syndrome according to any one of items 1a to 14a, wherein a dose of the active ingredient is 125 to 3000 mg/day/person.

### Advantageous Effects of Invention

The therapeutic agent for blood-brain barrier dysfunction syndrome according to the present invention is effective for enhancing blood-brain barrier functions including tight junction between cerebrovascular endothelial cells, transcellular transport, etc., thereby preventing, suppressing, or ameliorating a disease or a symptom attributed to blood-brain barrier dysfunction.

### BRIEF DESCRIPTION OF DRAWINGS

FIG. 1 is a schematic diagram of an in vitro BBB model test.

FIG. 2 is a graph showing that the medicament of the present invention suppressed the permeability of albumin.

FIG. 3 is a graph showing that the medicament of the present invention suppressed the permeability of fluorescein sodium.

FIG. 4 is a graph showing that the medicament of the present invention suppressed the hyperpermeability of fluorescein sodium induced by CsA in the blood-brain barrier.

FIG. 5 is a graph showing that the medicament of the present invention suppressed the hyperpermeability of fluorescein sodium induced by t-PA in the blood-brain barrier.

FIG. 6 is a graph showing that the medicament of the present invention suppressed the hyperpermeability of albumin induced by t-PA in the blood-brain barrier.

FIG. 7 is a photograph of the brain of a cerebral infarction mouse model.

FIG. 8 is a graph showing that the medicament of the present invention suppressed the hyperpermeability of fluorescein sodium induced by t-PA in the blood-brain barrier of a cerebral infarction mouse model.

FIG. 9 is a graph showing that the medicament of the present invention suppressed the hyperpermeability of albumin induced by t-PA in the blood-brain barrier of a cerebral infarction mouse model.

### DESCRIPTION OF EMBODIMENTS

The therapeutic agent for blood-brain barrier dysfunction syndrome according to the present invention enhances blood-brain barrier functions including tight junction between cerebrovascular endothelial cells, transcellular transport, etc. As a result, the medicament or the pharmaceutical composition of the present invention prevents, suppresses, or ameliorates the pathological conditions caused by blood-brain barrier dysfunction, thereby contributing to the prevention, suppression, or amelioration of a disease or a symptom attributed to blood-brain barrier dysfunction, such as the diseases described above.

Examples of an active ingredient in the therapeutic agent for blood-brain barrier dysfunction syndrome according to the present invention include biguanides agents such as metformin and buformin, and pharmaceutically acceptable salts thereof.

When the therapeutic agent for blood-brain barrier dysfunction syndrome according to the present invention comprises a pharmaceutically acceptable salt of a biguanides agent as an active ingredient, the salt is not limited by a type. Any inorganic or organic acid salt can be used. Examples of an inorganic acid for the inorganic acid salt include hydrochloric acid, sulfuric acid, nitric acid, phosphoric acid, and boric acid. Examples of a salt for the organic acid salt include formic acid, acetic acid, propionic acid, lactic acid, benzoic acid, oxalic acid, succinic acid, fumaric acid, maleic acid, malic acid, tartaric acid, citric acid, methanesulfonic acid, and benzenesulfonic acid. Preferable examples of the salt include hydrochloride. In this context, metformin hydrochloride and buformin hydrochloride are commercially available as hypoglycemic agents.

The biguanides agent of the present invention or the pharmaceutically acceptable salt thereof is not limited by a production method and can be produced by a method known in the art.

Alternatively, the biguanides agent of the present invention or the pharmaceutically acceptable salt thereof can be made into a preparation according to a routine method, either alone or in combination with a pharmaceutically acceptable pharmaceutical carrier known in the art.

The therapeutic agent for blood-brain barrier dysfunction syndrome according to the present invention is not limited by an administration method. Examples thereof include oral administration, administration through intravenous injection, administration through intramuscular injection, administration through intraperitoneal injection, administration through hypodermic or intradermal injection, intrarectal administration, transmucosal administration, and administration via the respiratory tract. Preferable examples of the administration method include oral administration.

The therapeutic agent for blood-brain barrier dysfunction syndrome according to the present invention is not limited by its dosage form. Examples of the dosage form for oral administration include tablets, granules, capsules, powders, solutions, suspensions, and syrups. Examples of the dosage form for parenteral administration include injections, intravenous drops, suppositories, and percutaneously absorbable formulations.

The pharmaceutical composition in any of the dosage forms described above can be produced using a pharmaceutically acceptable pharmaceutical carrier known in the art. For example, an excipient, a binder, a disintegrant, a lubricant, a stabilizer, a coloring agent, and a corrigent can be used.

Examples of the excipient include acrylic acid starch, gum arabic, lactose, corn starch, saccharose, glucose, sorbitol, crystalline cellulose, silicon dioxide calcium silicate, and magnesium silicate. Examples of the binder include polyvinyl alcohol, calcium citrate, carboxyvinyl polymer, carboxymethylethylcellulose, polyvinyl ether, methylcellulose, gum arabic, tragacanth, gelatin, shellac, hydroxypropylcellulose, hydroxypropylmethylcellulose, ethylcellulose, dextrin, sodium polyphosphate, and pectin. Examples of the disintegrant include: disintegrants called super disintegrants such as crospovidone, croscarmellose sodium, and carmellose calcium; hydroxypropylcellulose; carboxymethyl starch sodium; and corn starch. Examples of the lubricant include magnesium stearate, talc, polyethylene glycol, aluminum stearate, lactose, magnesium carbonate, carmellose calcium, carmellose sodium, and hydrogenated plant oil. Examples of the stabilizer include sodium edetate, sodium sulfite, butylhydroxyanisole, and butylhydroxytoluene. Examples of the coloring agent include: caramel; edible dyes or edible lake dyes such as Food Yellow No. 5, Food Red No. 2, and Food Blue No. 2; and iron red. Examples of the corrigent include hydrochloric acid, orange oil, fennel, cacao powder, menthol, aromatic acid, peppermint oil, and cinnamon bark oil. The tablets or the granules may be sugar-coated, gelatin-coated, or coated in any other manner, if necessary.

The medicament or the pharmaceutical composition according to the present invention can be administered to a human at a dose of 125 to 3000 mg/day/person in terms of metformin hydrochloride. The dose may be, for example, 250 mg/day/person, 500 mg/day/person, 750 mg/day/person, 850 mg/day/person, 1500 mg/day/person, 1700 mg/day/person, 2250 mg/day/person, or 2550 mg/day/person.

As mentioned above, blood-brain barrier dysfunction causes the onset of various pathological conditions or the exacerbation or recurrence of the pathological conditions and is predisposed to, for example, the onset, worsening, or recurrence of a disease related to the blood-brain barrier dysfunction. For example, sepsis is thought to cause a decline in blood-brain barrier functions, which in turn causes transport of neurotoxins from blood into the brain, resulting in encephalopathy (Non Patent Literature 3). Also, the increased expression of adhesion molecules on cerebrovascular endothelial cells constituting the blood-brain barrier is thought to allow leukocytes to easily permeate the blood-brain barrier, resulting in the progression of multiple sclerosis (Non Patent Literatures 3 and 4). In addition, a blood-brain barrier dysfunction is considered to allow amyloid beta protein (Aβ) to accumulate in the brain, resulting in the progression of Alzheimer's disease (Non Patent Literature 3).

Thus, the agent for enhancing a blood-brain barrier function according to the present invention is useful in the prevention, suppression, or amelioration of the diseases or the symptoms attributed to blood-brain barrier dysfunction.

For example, the therapeutic agent for blood-brain barrier dysfunction syndrome according to the present invention can be used for preventing or suppressing worsening or recurrence of cerebral infarction. The therapeutic agent for blood-brain barrier dysfunction syndrome according to the present invention can also be used for preventing, suppressing, or ameliorating cerebral edema associated with cerebral infarction. The therapeutic agent for blood-brain barrier dysfunction syndrome according to the present invention can also be used for preventing, suppressing, or ameliorating cerebral edema associated with cerebral trauma. The therapeutic agent for blood-brain barrier dysfunction syndrome according to the present invention can also be used for preventing, suppressing, or ameliorating encephalopathy associated with sepsis. The therapeutic agent for blood-brain barrier dysfunction syndrome according to the present invention can also be used for preventing, suppressing, or ameliorating leukocyte infiltration into the brain in multiple sclerosis. The therapeutic agent for blood-brain barrier dysfunction syndrome according to the present invention can also be used for preventing, suppressing, or ameliorating accumulation of amyloid beta protein (Aβ) in the brain in Alzheimer's disease.

In an alternative embodiment of the present invention, the therapeutic agent for blood-brain barrier dysfunction syndrome according to the present invention can be used for preventing or suppressing drug transport into the brain. The drug may be a drug that possesses the unwanted effects on an animal or human central nervous system. Alternatively, the drug may be a drug that possesses the unwanted effects on an animal or human central nervous system. In such a case, the medicament or the pharmaceutical composition of the present invention may be used even for suppressing transport of the drug into the brain in an amount more than expected.

In addition, the medicament or the pharmaceutical composition of the present invention can be used for preventing or suppressing the drug transport into the brain, thereby preventing, suppressing, or ameliorating an adverse symptom that affects the animal or human central nervous system.

For example, an immunosuppressive drug (cyclosporine, tacrolimus, etc.)-induced blood-brain barrier dysfunctions and following blood-brain barrier hyperpermeability are known to be responsible for adverse reactions such as tremor, convulsion, leukoencephalopathy, and headache that result from central adverse reactions (Non Patent Literatures 5 to 12). The present invention can suppress such a decline in blood-brain barrier functions or enhance or reconstruct the blood-brain barrier functions, thereby preventing, suppressing, or ameliorating the adverse symptoms.

The present invention can also prevent, suppress, or ameliorate a thrombolytic drug (tissue plasminogen activator, etc.)-induced blood-brain barrier dysfunctions. The present invention can also prevent, suppress, or ameliorate hemorrhagic infarction associated with the blood-brain barrier dysfunctions. The present invention can also be used for expanding a therapeutic window during the application of the thrombolytic drug to the treatment of cerebral infarction. Therefore, the present invention can be used as a therapeutic agent for cerebral infarction in combination with the thrombolytic drug.

In addition, the present invention can prevent, suppress, or ameliorate sleepiness caused by antihistamine drugs, disturbance of consciousness, abnormal behavior, delirium, hallucination, delusion, convulsion, etc., caused by Tamiflu, convulsion or epilepsy caused by interferon, infliximab, antidepressants, or new quinolone antimicrobial agents, and leukoencephalopathy caused by carmofur, tegafur, or fluorouracil.

In an alternative embodiment of the present invention, the therapeutic agent for blood-brain barrier dysfunction syndrome according to the present invention or the medicament or the pharmaceutical composition of the present invention can be used for preventing, suppressing, or ameliorating a decline in blood-brain barrier functions.

For example, diabetes mellitus, obesity, or hypertension is known to possibly cause a decline in blood-brain barrier functions (Non Patent Literatures 14 and 15). Therefore, the therapeutic agent for blood-brain barrier dysfunction syndrome according to the present invention can be used for preventing, suppressing, or ameliorating a decline in blood-brain barrier functions induced by diabetes mellitus, obesity, hypertension, or the like. The prevention, suppression, or amelioration of the blood-brain barrier dysfunctions leads to prevention of worsening or recurrence of cerebral infarction, prevention or suppression of drug transfer to the brain, etc.

Meanwhile, such a decline in blood-brain barrier functions may cause onset or worsening of diabetes mellitus and/or obesity. Therefore, the therapeutic agent for blood-brain barrier dysfunction syndrome according to the present invention can also be used for preventing, suppressing, or ameliorating the diseases attributed to a decline in blood-brain barrier functions.

In addition, the biguanides agent or the salt thereof, or the substance or the composition comprising it as an active ingredient can also be used as a reagent for animal tests for the effects or uses described above. The animal can be selected from, for example, cyprinodonts, frogs, lizards, chickens, mice, rats, guinea pigs, hamsters, rabbits, dogs, cats, sheep, pigs, goats, cow, and monkeys.

### EXAMPLES

The present invention will be described in more detail with reference to Examples below. However, the present invention is not limited to these Examples by any means.

### Example 1

### Example 1-1

### Preparation of In Vitro BBB Model

Cerebrovascular endothelial cells (rat brain endothelial cells: RBECs) were isolated according to Reference 1 shown below. The details are as follows:

A 3-week-old Wistar rat was anesthetized with ether and then decapitated. The cerebrum was excised and placed in a dish on ice. After removal of the meninges, the cerebral cortex was cut finely in a dish on ice. The slices were enzymatically treated by shaking (200 rpm) at 37° C. for 1.5 hours with collagenase (CLS2) (1 mg/ml; Worthington Biochemical Corp.) and deoxyribonuclease I (50 units/ml; Sigma-Aldrich Corp.). After centrifugation, 20% bovine serum albumin (BSA)-DMEM was added to the obtained pellet, and the mixture was centrifuged (1000×g, 20 minutes) to remove neurons and glia cells. Then, the residue was enzymatically treated by shaking (200 rpm) at 37° C. for 30 minutes with collagenase/dispase (1 mg/ml; Boehringer Mannheim K.K.) and deoxyribonuclease I (50 units/ml; Sigma-Aldrich Corp.). After centrifugation, the obtained pellet was suspended in a small amount of DMEM and added to a 33% Percoll (GE Healthcare Japan Corp.) solution with a density gradient formed in advance at 30000×g at 4° C. for 1 hour. The mixture was centrifuged to isolate brain capillary fractions.

The brain capillary fractions thus isolated were cultured at 37° C. under 5% CO₂/95% atmosphere in DMEM/F12 containing 10% plasma derived serum (PDS), 50 μg/mL gentamicin, 1 mM L-glutamine, 1 mg/mL heparin, 1.5 ng/mL bFGF, 5 μg/mL insulin, 5 μg/mL transferrin, 5 ng/mL selenium, and 4 μg/mL puromycin (RBEC culture solution I) using a culture dish coated with collagen and fibronectin. Forty eight hours later, the medium was replaced with RBEC culture solution I except for puromycin (RBEC culture solution II) The resultant cells were further cultured to obtain cerebrovascular endothelial cells (RBECs).

A monolayer in vitro BBB model, which was a monolayer RBEC culture system, was prepared from the RBECs thus obtained using Transwell (registered trademark) (24-well type, Corning Costar Corp., MA).

Specifically, Transwell insert (12-well type, Corning Costar Corp., MA) with a polycarbonate membrane (0.4 μm pore size) coated with collagen and fibronectin was placed in each well of a 24-well culture plate (Corning Costar Corp., MA). RBECs (5.0×10⁴ cells/well) were inoculated in the insert (monolayer). On the next day of RBEC inoculation, the solution was replaced with RBEC culture solution II containing hydrocortisone (500 nM). Two days later, a completed in vitro BBB model was used in experiments.

### Example 1-2

### Permeability Experiment

The influence of metformin on BBB functions was confirmed with the permeability coefficients of fluorescein sodium (Na—F) (Sigma-Aldrich Corp., St. Louis, Mo.) and Evans blue-albumin (albumin) (Evans blue; Sigma-Aldrich Corp., E2129, and bovine serum albumin; Sigma-Aldrich Corp., A7906) as an index.

In this context, a vascular relaxation factor adrenomedullin enhances blood-brain barrier functions, thereby suppressing the permeation of fluorescein sodium in vitro and in vivo (Non Patent Literature 16 and the unpublished data of the present inventor). Specifically, in vitro and in vivo tests on the permeation of fluorescein sodium have a positive relationship.

The test samples used were 0.1 mM metformin (metformin hydrochloride; Sigma-Aldrich Corp., D15, 095-9), 0.5 mM metformin (the same as above), and 1 mM metformin (the same as above) each dissolved in RBEC culture solution II except for PDS. The control group used was RBEC culture solution II except for PDS. The culture solution in the in vitro BBB model completed as above was fully removed and instead replaced with each test sample. 24 hours later, the permeability test was conducted.

A physiological buffer (0.1 ml) containing fluorescein sodium (100 μg/ml) or albumin (0.04 g/ml) was added to the vascular side of the insert. 10, 20, 30, 60, 120, and 180 minutes later, a sample (0.4 ml) was collected from each well (brain side). A fresh physiological buffer was added thereto in the same amount as above. The fluorescence intensity of fluorescein sodium was measured (excitation wavelength: 485 nm, fluorescence wavelength: 530 nm) using a fluorescence plate reader (CytoFluor (registered trademark) Series 4000, PerSeptive Biosystems, Inc., Framingham, Mass.). The absorption intensity of albumin (wavelength: 630 nm) was measured using an absorption plate reader (Sunrise (registered trademark), TECAN Group Ltd., Mannedorf, Switzerland). The concentrations of fluorescein sodium and albumin were calculated from calibration curves.

Clearance and permeability coefficients (P) were calculated according to References 1 and 2 shown below. The clearance was defined as the amount (indicated by μL) of fluorescein sodium or albumin delivered from the chamber on the vascular side to the chamber on the brain parenchyma side and calculated according to the following expression from the initial concentration [C]_{L} of fluorescein sodium or albumin placed in the vascular side and the final concentration [C]_{A} of fluorescein sodium or albumin delivered to the brain parenchyma side:

Clearance (μl)=[C]_{A}×V_{A}/[C]_{L}

(V_{A}: Volume (1.5 ml) of the chamber on the brain parenchyma side)

The permeability coefficient P (cm/min) was determined according to the following expression:

1/PSₐₚₚ=1/PS_{membrane}+1/PSₜᵣₐₙₛ

PS represents the slope of a line on which clearance was plotted against time and is indicated by (Permeability coefficient)×(Surface area of the membrane). Pₐₚₚ represents an apparent permeability coefficient. Pₜᵣₐₙₛ represents a true permeability coefficient. P_{membrane} represents a permeability coefficient derived from only the membrane of the chamber.

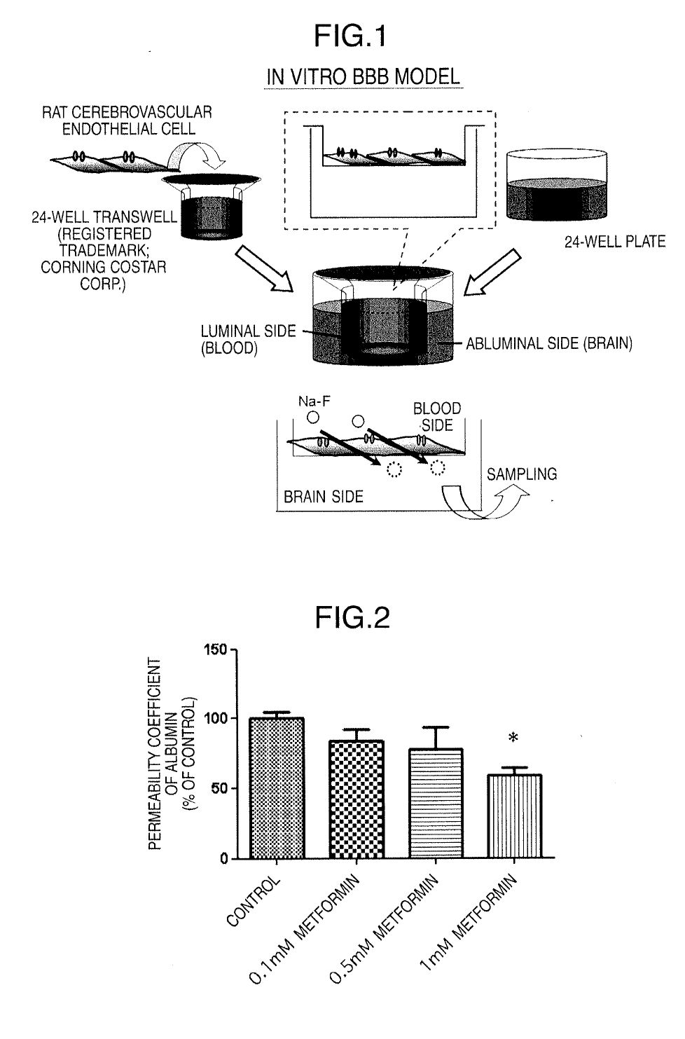

A schematic diagram of this test is shown in FIG. 1.

The test was conducted with reference to the literatures described above and the following references:
[Reference 1]
Isobe, I., Watanabe, T., Hazemoto, N., Yamagata, K., Ueki, T., Nakanishi, K., Asai, K., Kato, T., “Astrocytic contributions to blood-brain barrier (BBB) formation by endothelial cells: a possible use of aortic endothelial cell for in vitro model”, Neurochemistry International, 1996, 28, p. 523-533
[Reference 2]
Dehouck, M.-P., Jolliet-Riant, P., Bree, F., Fruchart J.-C., Cecchelli, R., Tillement, J.-P., “Drug transfer across the blood-brain barrier: correlation between in vitro and in vivo models”, Journal of Neurochemistry, 1992, 58, p. 1790-1797
[Reference 3]
Hayashi Y., Nomura M., Yamagishi S., Harada S., Yamashita J., Yamamoto H., “Induction of various blood-brain barrier properties in non-neural endothelial cells by close apposition to co-cultured astrocytes”, Glia, 1997, 19, p. 13-26
[Reference 4]
Takata F, Sumi N, Nishioku T, Harada E, Wakigawa T, Shuto H, Yamauchi A, Kataoka Y., “Oncostatin M induces functional and structural impairment of blood-brain barriers comprised of rat brain capillary endothelial cells”, Neuroscience Letters, 2008, 441(2), p. 163-166

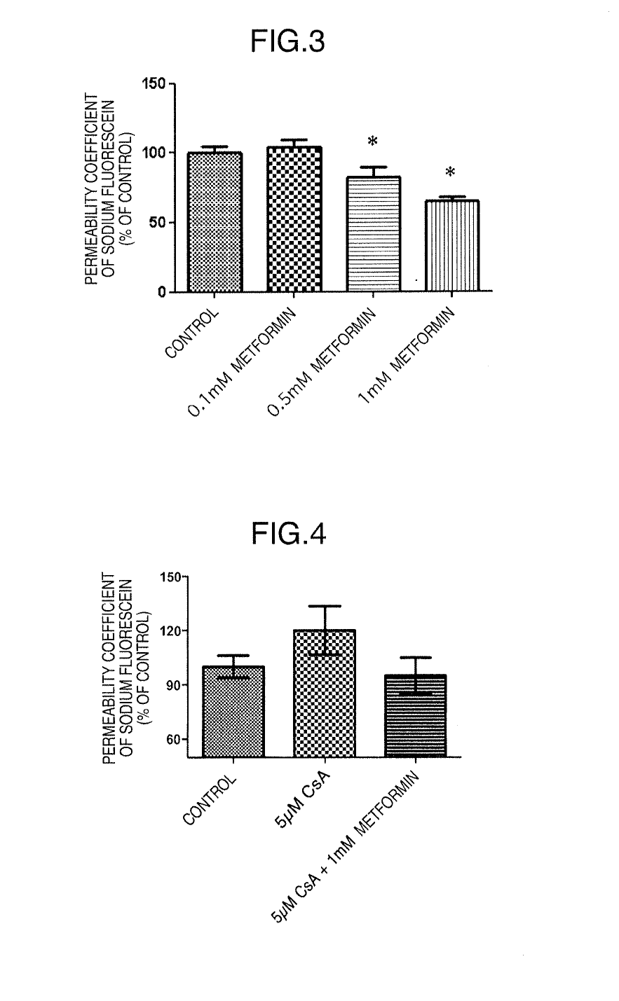

Test results obtained using each concentration (0.1 mM, 0.5 mM, and 1 mM) of metformin with test results (permeability coefficient) of the control group as 100% are shown in FIGS. 2 and 3. FIG. 2 shows the results of determining the permeability coefficient of albumin. FIG. 3 shows the results of determining the permeability coefficient of fluorescein sodium.

In the test to determine the permeability coefficient of albumin, the one-way ANOVA method of 4 groups produced a significant difference with a significance probability P of 0.0009 and a significance level of 0.05. A significance test further conducted using a Dunnett's multiple comparison test method with a significance level of 0.05 between the control group and each metformin group resulted in a significant difference of the 1 mM metformin group. This group is marked with * in FIG. 2.

In the test to determine the permeability coefficient of fluorescein sodium, the one-way ANOVA method of 4 groups produced a significant difference with a significance probability P less than 0.0001 and a significance level of 0.05. A significance test further conducted using a Dunnett's multiple comparison test method with a significance level of 0.05 between the control group and each metformin group resulted in a significant difference of the 0.5 mM metformin group and the 1 mM metformin group. These groups are marked with * in FIG. 3.

As shown in FIGS. 2 and 3, metformin significantly suppressed the permeability of albumin or a tight junction marker fluorescein sodium from blood side into brain side, compared with the control group.

### Example 2

### Protective Effect of Metformin on Blood-Brain Barrier Dysfunction Induced by Cyclosporin A (CsA)

### Central Adverse Reaction-Inducing Drug

An immunosuppressive drug cyclosporin A (CsA) is a beneficial medicament that improves the success rate of organ transplantation. On the other hand, this drug has many adverse reactions. Its administration must be discontinued when central adverse reactions such as tremor or convulsion occur. We have previously revealed that the increased permeability of CsA into the brain in association with CsA-induced decline in blood-brain barrier functions is involved in the occurrence of central adverse reactions. Thus, metformin was examined for its effect on the CsA-induced decline in blood-brain barrier functions.

### Example 2-1

An in vitro BBB model was prepared according to Example 1-1.

### Example 2-2

A permeability experiment was conducted in the same way as in Example 1-2 except that a different method for drug stimulation was used.

The test samples used were CsA dissolved in ethanol and 1 mM metformin (metformin hydrochloride; Sigma-Aldrich Corp., D15, 095-9) dissolved in RBEC culture solution II except for PDS. The control group used was ethanol and RBEC culture solution II except for PDS. The culture solution in the in vitro BBB model completed as above was fully removed and instead replaced with each test sample. Twenty four hours later, the permeability test was conducted.

The results of this test are shown in FIG. 4. As shown in the diagram, the loading of CsA increased the permeability of fluorescein sodium, whereas the combined use with metformin suppressed the CsA-induced decline in blood-brain barrier functions, thereby significantly suppressing the increase in the permeability of fluorescein sodium.

### Example 3

### In Vitro Effect of Metformin on Blood-Brain Barrier Dysfunction Induced by Tissue Plasminogen Activator (t-PA)

### Central Adverse Reaction-Inducing Drug

A “tissue plasminogen activator (t-PA)”, a thrombolytic drug for cerebral infarction, exhibits very favorable effects when intravenously administered by 3 to 4.5 hours from onset. The thrombolytic therapy using t-PA, however, has disadvantages. One of the disadvantages is a therapeutic window as very short as 3 hours after cerebral infarction (Non Patent Literature 2) because blood-brain barrier dysfunction associated with use of t-PA under ischemic conditions may be complicated by hemorrhagic infarction (Non Patent Literatures 17 and 18). Thus, a “t-PA-induced BBB dysfunction model” was prepared by the loading of ischemia and t-PA onto an in vitro BBB model. Metformin was examined for its protective effect on blood-brain barrier functions.

### Example 3-1

An in vitro BBB model was prepared according to Example 1-1.

### Example 3-2

A permeability experiment was conducted in the same way as in Example 1-2 except that a different method for drug stimulation was used.

The test samples used were a t-PA injection (Activacin; Kyowa Hakko Kirin Co., Ltd.) and 1 mM metformin (metformin hydrochloride; Sigma-Aldrich Corp., D15, 095-9) dissolved in RBEC culture solution I except for PDS. The control group used was RBEC culture solution I except for PDS. The culture solution in the in vitro BBB model completed as above was fully removed and instead replaced with each test sample. The resulting sample was left for 48 hours under 95% N₂/5% CO₂ (ischemia) conditions. Then, the permeability test was conducted.

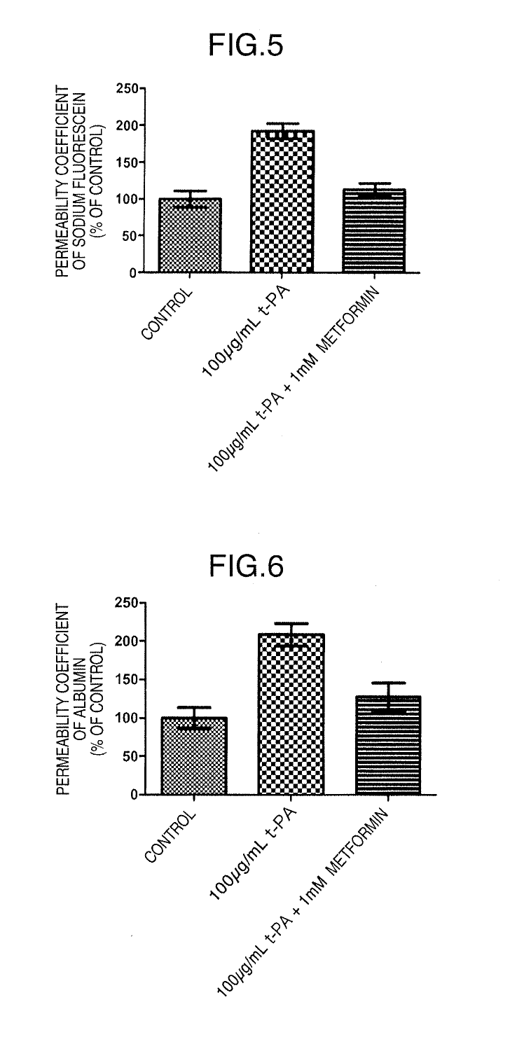

The results of this test are shown in FIGS. 5 and 6. As shown in both the diagrams, the loading of t-PA under ischemic conditions increased the permeability of fluorescein sodium and albumin (Evans blue-albumin: EBA), whereas the combined use with metformin suppressed the t-PA-induced decline in blood-brain barrier functions, thereby significantly suppressed the increase in the permeability of fluorescein sodium and albumin.

### Example 4

### In Vivo Effect of Metformin on Blood-Brain Barrier Dysfunction Induced by t-PA

### Central Adverse Reaction-Inducing Drug

### Example 4-1

### Preparation of In Vivo t-PA-Induced BBB Dysfunction Model

Both common carotid arteries of each ddY mouse were ligated (2VO) for 30 minutes to prepare a cerebral infarction mouse model. After reopening of blood vessels, t-PA (10 mg/kg) was administered to the subclavian vein of the resulting mouse. The mouse was left for 24 hours and used as a t-PA-induced BBB dysfunction mouse model.

Metformin was administered at a dose of 100 mg/kg concurrently with t-PA. The control group used was a drug-unadministered cerebral infarction mouse model.

### Example 4-2

### Evaluation of BBB Function

24 hours later after the administration of t-PA or the administration of t-PA and metformin, 200 μL of a mixed solution of fluorescein sodium (Na—F) (6 mg/mL) and Evans blue (EB) (20 mg/mL) was administered to the subclavian vein of the mouse. The mouse was left for 60 minutes. Then, the whole brain was excised. BBB functions were evaluated using a photograph of the whole brain and the concentrations of Na—F and EB in whole brain homogenates.

### Results

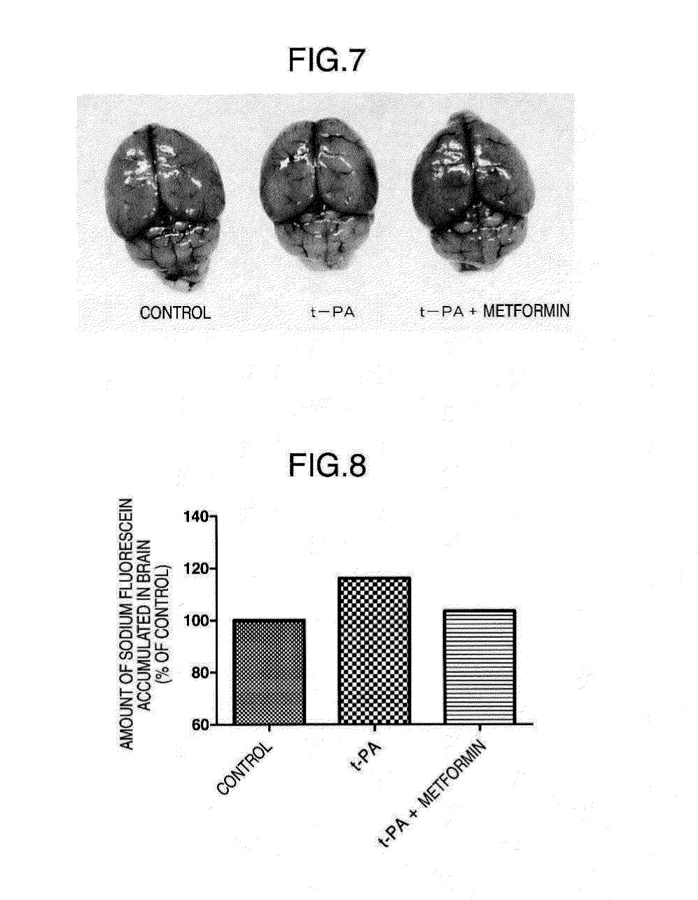

When t-PA was loaded onto the cerebral infarction mouse model, its brain was stained blue, demonstrating that EB was leaked from blood vessels into the whole brain (FIG. 7, middle). The combined use thereof with metformin lightened the blue color of the brain (FIG. 7, right), demonstrating that EB leakage from blood vessels was suppressed. This indicates that the combined use with metformin suppressed BBB dysfunction caused by the loading of t-PA.

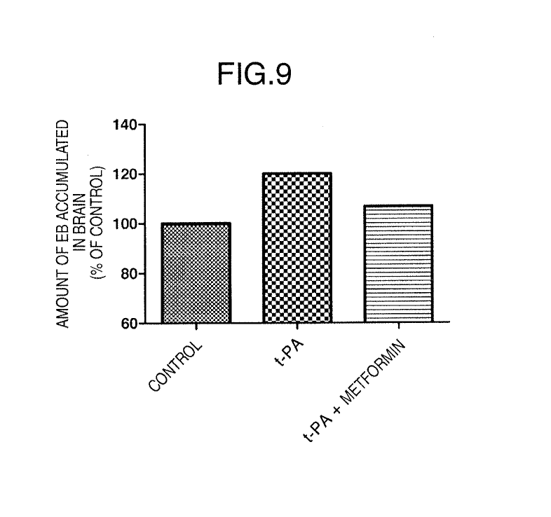

The loading of t-PA onto the cerebral infarction mouse model increased the amounts of Na—F and EB transported into the brain by 16% and 20%, respectively (FIGS. 8 and 9). The combined use with metformin suppressed the increase in the transport of Na—F and EB by 78% and 66%, respectively (FIGS. 8 and 9).

1-21. (canceled)

22. A medicament for enhancing a blood-brain barrier function, comprising a biguanides agent or a pharmaceutically acceptable salt thereof as an active ingredient.

23. The medicament according to claim 22, wherein said enhancement of the blood-brain barrier function is prevention, suppression, or promotion of the suppression of a decline in the blood-brain barrier function.

24. The medicament according to claim 23, wherein said decline in the blood-brain barrier function is attributed to a disease selected from the group consisting of diabetes mellitus, obesity, hypertension, cerebral infarction, cerebral trauma, sepsis, multiple sclerosis, Alzheimer's disease, and combination thereof, or attributed to a substance inducing the central adverse reactions.

25. The medicament according to claim 24, wherein said substance inducing the central adverse reactions is an immunosuppressive drug or a thrombolytic drug.

26. The medicament according to claim 22, wherein the medicament is used in combination with a drug for preventing, suppressing, or ameliorating cerebral edema associated with cerebral infarction or cerebral trauma.

27. The medicament according to claim 22, wherein the medicament prevents or suppresses drug transport into the brain.

28. The medicament according to claim 27, wherein said drug is a drug that possesses the unwanted effects on an animal or human central nervous system.

29. The medicament according to claim 27, wherein the medicament prevents or suppresses said drug transport into the brain, thereby preventing, suppressing, or ameliorating an adverse reaction that affects the animal or human central nervous system.

30. The medicament according to claim 29, wherein said adverse reaction is selected from tremor, convulsion, leukoencephalopathy, headache, sleepiness, disturbance of consciousness, abnormal behavior, delirium, hallucination, delusion, epilepsy, cerebral infarction, cerebral hemorrhage and combination thereof.

31. The medicament according to any one of claim 27, said drug is an immunosuppressive drug.

32. The medicament according to claim 25, said immunosuppressive drug is cyclosporin A (CsA).

33. The medicament according to claim 26, wherein said drug is a thrombolytic drug.

34. The medicament according to claim 25 wherein said thrombolytic drug is a tissue plasminogen activator (t-PA).

35. The medicament according to claim 22, wherein said biguanides agent is metformin or buformin.

36. The medicament according to claim 35, wherein said biguanides agent or the pharmaceutically acceptable salt thereof is metformin hydrochloride or buformin hydrochloride.

37. The medicament according to claim 36, wherein said biguanides agent or the pharmaceutically acceptable salt thereof is metformin hydrochloride.

38. A medicament for enhancing a blood-brain barrier function, comprising a biguanides agent or a pharmaceutically acceptable salt thereof as an active ingredient, used in combination with cyclosporin A (CsA), and wherein said biguanides agent or the pharmaceutically acceptable salt thereof is metformin hydrochloride.

39. A medicament for enhancing a blood-brain barrier function, comprising a biguanides agent or a pharmaceutically acceptable salt thereof as an active ingredient, used in combination with a tissue plasminogen activator (t-PA), and wherein said biguanides agent or the pharmaceutically acceptable salt thereof is metformin hydrochloride.

40. The medicament according to claim 22, wherein a dose of the active ingredient is 125 to 3000 mg/day/person.

41. A method for preventing, suppressing, or ameliorating a disease, wherein the disease is selected from the group consisting of tremor, convulsion, leukoencephalopathy, headache, sleepiness, disturbance of consciousness, abnormal behavior, delirium, hallucination, delusion, epilepsy, cerebral infarction, cerebral hemorrhage, cerebral trauma, sepsis, Alzheimer's disease, and combination thereof, the method comprising the step of administering a medicament according to claim 22 to an individual in need thereof.

42. A method for treating blood-brain barrier dysfunction syndrome, comprising the step of administering a medicament comprising a biguanides agent or a pharmaceutically acceptable salt thereof as an active ingredient to an individual in need thereof to enhance a blood-brain barrier function.
